(19) United States
(12) Patent Application Publication
(10) Pub. No.: US 2021/0197022 A1
(43) Pub. Date: Jul. 1, 2021

(54) EVALUATION METHOD, MODEL ESTABLISHING METHOD, TEACHING DEVICE, SYSTEM, AND ELECTRICAL APPARATUS

(71) Applicant: AI4FIT INC., Wilmington, DE (US)

(72) Inventor: Weijie LIU, Wilmington, DE (US)

(73) Assignee: AI4FIT INC., Wilmington, DE (US)

(21) Appl. No.: 16/833,370

(22) Filed: Mar. 27, 2020

(30) Foreign Application Priority Data
Dec. 31, 2019 (CN) 201911415218.2

(51) Int. Cl. A63B 24/00; A63B 71/06; A61B 5/024; G06F 3/01; G06T 7/73

(52) U.S. Cl. CPC A63B 24/0062; A63B 24/0075; A63B 71/0622; A63B 2220/05; G06F 3/017; G06T 7/74; A63B 2024/0065; A61B 5/024

(57) ABSTRACT

The present disclosure provides an evaluation method, a model establishing method, a teaching device, system, and an electrical apparatus. The method includes: collecting image information containing a user's image; acquiring an action model corresponding to training actions that the user refers to; performing evaluation on action information of at least one part of the user's body in the image information by using the action model to obtain an evaluation result; outputting the evaluation result on the action information of at least one part of the user's body.

### TECHNICAL FIELD

The present disclosure relates to a field of computer, and particularly relates to an evaluation method, a model establishing method, a teaching device, system, and an electrical apparatus.

### BACKGROUND

In the prior art, when a user is taking exercises, he or she carries out a training alone or by watching a training video, and thus it is difficult for the user to accurately determine whether his own movement is correct, and generally the user requires a teaching from a fitness coach. The user may require a professional personal trainer to make a one-on-one and on-the-spot teaching. Users can make a face-to-face personal communication with the personal trainer. This way requires users to make an appointment with the trainer in advance and Both of them to go to the gym for training at the same time, which may waste a lot of time and have a high cost for fitness.

### BRIEF SUMMARY

In view of this, the present disclosure provides an evaluation method, a model establishing method, a teaching device, system, and an electrical apparatus to solve the technical problem that a user has to waste a lot of time and pay a lot for professional teaching on the user's exercise.

In one embodiment of the present disclosure, an evaluation method is provided. The method includes: collecting image information containing a user's image; acquiring an action model corresponding to training actions that the user refers to; performing evaluation on action information of at least one part of the user's body in the image information by using the action model to obtain an evaluation result; outputting the evaluation result on the action information of at least one part of the user's body.

In another embodiment of the present disclosure, a method for establishing a model is provided. The method may include: obtaining a video of a training project; processing the video by performing decomposition on the training actions to obtain frames corresponding to decomposed actions; establishing an action model based on the frames corresponding to the decomposed actions, wherein the action model is used to perform evaluation on action information of at least one part of a user's body in collected image information containing a user's image.

In another embodiment of the present disclosure, a teaching device is provided. The teaching device may include: a collecting means configured to collect image information containing a user's image; a processor configured to acquire an action model corresponding to training actions that the user refers to; perform evaluation on action information of at least one part of the user's body in the image information by using the action model to obtain an evaluation result; outputting the evaluation result to an outputting means.

In another embodiment of the present disclosure, a teaching system is provided. The teaching system may include: a teaching device configured to collect image information containing a user's images; and sending the image information to a server; a server configured to acquire an action model corresponding to training actions that the user refers to; perform evaluation on action information of at least one part of the user's body in the image information by using the action model to obtain an evaluation result; send the evaluation result to the teaching device, wherein the teaching device is further configured to output the evaluation result on the action information of at least one part of the user's body.

In another embodiment of the present disclosure, an electrical apparatus is provided. The electrical device may include: a memory and a processor, wherein the memory is configured to store a program; the processor is coupled to the memory, and is configured to execute the program stored in the memory, to: collect image information containing a user's image; acquire an action model corresponding to training actions that the user refers to; perform evaluation on action information of at least one part of the user's body in the image information by using the action model to obtain an evaluation result; output the evaluation result on the action information of at least one part of the user's body.

In another embodiment of the present disclosure, an electrical apparatus is provided. The electrical apparatus includes: a memory and a processor, wherein the memory is used to store a program; the processor is coupled to the memory, and is configured to execute the program stored in the memory, to: obtain a video of a training project; process the video by performing decomposition on the training actions to obtain frames corresponding to decomposed actions; establish an action model based on the frames corresponding to the decomposed actions, wherein the action model is used to perform evaluation on action information of at least one part of a user's body in collected image information containing a user's image.

The technical solution provided in the embodiments of the present disclosure may achieve an evaluation on the action information of a user based on standard action model automatically by collecting image information containing a user's image; acquiring an action model corresponding to training actions that the user refers to; performing evaluation on action information of at least one part of the user's body in the image information by using the action model to obtain an evaluation result; outputting the evaluation result on the action information of at least one part of the user's body. The evaluation method of the present disclosure may perform detailed evaluation on details of actions of a user's body so that the user may acknowledge whether or not the details of actions are accurate and the time for exercising may be efficiently used and the cost of exercising may be lowered.

The above description is merely a brief introduction of the technical solutions of the present disclosure, so that the technical means of the present disclosure may be clearly understood, and implemented according to the description of the specification, and the above and other technical objects, features and advantages of the present disclosure may be more obvious based on the embodiments of the present disclosure as follows.

### BRIEF DESCRIPTION OF THE DRAWINGS

Drawings needed in the description of the embodiments and the prior art shall be explained below, so as to explain the technical solutions in the embodiments of the present invention and the prior art more clearly. It is obvious that the drawings explained below are merely some embodiments of the present invention, and a person of ordinary skill in the art may obtain other drawings according to these drawings without making an inventive effort.

FIG. 1 is a schematic flowchart of an evaluation method according to an embodiment of the present disclosure;

FIG. 2 is a schematic flowchart of a model establishing method according to an embodiment of the present disclosure;

FIG. 3 is a schematic structural diagram of a teaching system according to an embodiment of the present disclosure;

FIG. 4 is a schematic flowchart of an evaluation method according to an embodiment of the present disclosure;

FIG. 5 is a schematic structural diagram of a teaching device according to an embodiment of the present disclosure;

FIG. 6 is a schematic structural diagram of an evaluation device according to an embodiment of the present disclosure;

FIG. 7 is a schematic structural diagram of a model establishing apparatus according to an embodiment of the present disclosure;

FIG. 8 is a schematic structural diagram of a teaching system according to an embodiment of the present disclosure;

FIG. 9 is a schematic structural diagram of an electrical apparatus according to an embodiment of the present disclosure;

FIG. 10 is a schematic structural diagram of an electrical apparatus according to an embodiment of the present disclosure.

### DETAILED DESCRIPTION

In order to make the objectives, technical solutions, and advantages of the embodiments of the present disclosure clearer, the technical solutions in the embodiments of the present disclosure will be clearly and completely described with reference to the drawings in the embodiments of the present disclosure. Obviously, the described embodiments are some of the embodiments of the present disclosure, not all of the embodiments. Based on the embodiments in the present disclosure, all other embodiments obtained by one skilled in the art without making creative efforts shall fall within the protection scope of the present disclosure.

The terms used in the embodiments of the present disclosure are only for the purpose of describing specific embodiments, and are not intended to limit the present disclosure. The singular forms “a”, “said” and “the” used in the examples of the present disclosure and the claims are also intended to include the plural form, unless the context clearly indicates other meanings. Generally, “a plurality of kinds” means at least two kinds are included, without excluding the case where at least one kind is included.

It should be understood that the term “and/or” used herein is merely an association relationship describing related objects, indicating that there can be three relationships. For example, A and/or B can indicate the following three situations: A alone, A and B, and B alone. In addition, the character “I” herein generally indicates that the related objects are in a relationship of “or”.

It should be understood that although the terms of first, second, third, etc. may be used to describe XXX in the embodiments of the present disclosure, these XXX should not be limited by these terms. These terms are only used to distinguish XXX from each other. For example, without departing from the scope of the embodiments of the present disclosure, the first XXX may also be referred to as the second XXX, and similarly, the second XXX may also be referred to as the first XXX. Depending on the context, the word of “if”, as used herein, can be interpreted as “at the time” or “when” or “in response to determining” or “in response to monitoring”. Similarly, depending on the context, the phrase of “if determined” or “if monitored (condition or event as stated)” can be interpreted as “when determined” or “in response to determining” or “when monitoring (condition or event as stated)” or “in response to monitoring (condition or event as stated)”.

It should also be noted that the terms of “including”, “containing” or any other variation thereof are intended to encompass non-exclusive inclusions, so that a product or system that includes a series of elements includes not only those elements but also other elements that are not explicitly listed, or elements that are inherent to this commodity or system. Without much limitation, the elements defined by the expression of “including a . . . ” does not exclude the existence of other same elements in the product or system including elements as stated.

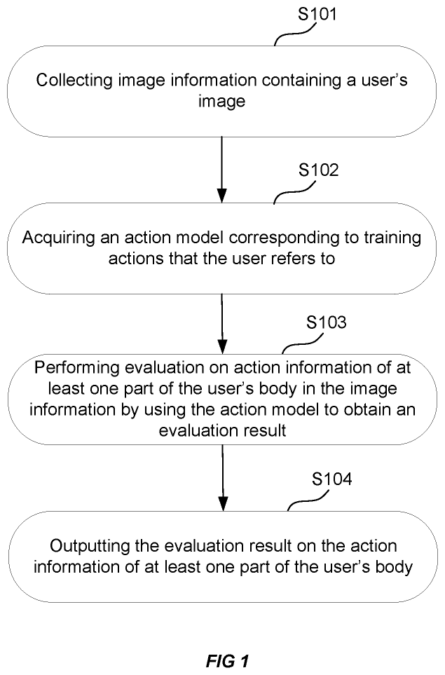

FIG. 1 is a schematic flowchart of an evaluation method according to an embodiment of the present disclosure. The execution subject of the method provided by the embodiments of the present disclosure may be a device, which may be, but is not limited to, a device incorporated in any terminals, such as a smartphone, a tablet computer, a PDA (Personal Digital Assistant), a smart TV, a laptop, a portable computer, desktop computer, and smart wearable device. As shown in FIG. 1, the evaluation method includes:

S101, Collecting image information containing a user's image.

S102, Acquiring an action model corresponding to training actions that the user refers to.

S103, Performing evaluation on action information of at least one part of the user's body in the image information by using the action model to obtain an evaluation result.

S104, Outputting the evaluation result on the action information of at least one part of the user's body.

In the above step of S101, the collected image information containing a user's image may be two-dimensional information or three-dimensional information. The camera may be used to capture the image information of the user during exercise. The user's exercise type may include yoga, Tai Chi, rehabilitation training, dance training, etc. For example, one camera may be provided facing the user directly, or two, three, or four cameras may be provided around the user, so as to collect image information containing an image of the user.

Furthermore, when taking image information containing a user's image, one or more cameras may be set at the taking location to take the image information.

In some embodiments of the present disclosure, a user may take exercise by referring to a video corresponding to standard training actions. In the present disclosure, an action model may be established for a video corresponding to a training action for a user to refer to. A decomposed action corresponding to a standard training action may correspond to an action model, which may be used to perform evaluation on the action information of at least one part of the user's body in the image information and obtain an evaluation result. The evaluation result may be represented by a score. The higher the score is, the closer to the standard action the action corresponding to the action information of at least one part of the user's in the image information is. Alternatively, the evaluation result may further include determination information indicating the action is right or wrong.

In some embodiments of the present disclosure, one part of the user's body may correspond to one action model, or a plurality of parts of the user's body may correspond to one action model. For example, taking yoga (action of cobra) as an example, the head may correspond to one action model, or the head, arms, and legs may correspond to one action model.

The action model in S102 described above may be a model obtained based on machine-learning technology, such as neural network learning model, which is a common machine-learning model. The action model may be obtained by performing learning on a lot of training samples. The action model may have an input of image information, and an output of an evaluation result on action information of at least one part of the user. Accordingly, the step of S102 may be: inputting the image information to the action model, running the action model to obtain an evaluation result on action information of at least one part of the user, such as an evaluation result of being correct or wrong.

The evaluation result obtained in the above step of S104 may be output in the following ways: announcing the evaluation result in voice, or displaying the evaluation result in text of prompt.

The technical solution provided in the embodiment of the present disclosure may achieve an evaluation on the action information of a user based on standard action model automatically by collecting image information containing a user's image; acquiring an action model corresponding to training actions that the user refers to; performing evaluation on action information of at least one part of the user's body in the image information by using the action model to obtain an evaluation result; outputting the evaluation result on the action information of at least one part of the user's body. The evaluation method of the present disclosure may perform detailed evaluation on details of actions of a user's body so that the user may acknowledge whether or not the details of actions are accurate and the time for exercising may be efficiently used and the cost of exercising may be lowered.

Of course, the action model in this embodiment may have an input of information on character point of human body extracted from image information, and an output of an evaluation result on action information of at least one part of the user. That is to say, in some embodiments, the “performing evaluation on action information of at least one part of the user's body in the image information by using the action model to obtain an evaluation result” in the above step of S103 may be implemented by the following steps:

S1001: Collecting body feature point information of the user from the image information.

S1002. Using the body feature point information as an input parameter of the action model, running the action model, and obtaining an evaluation result of action information of at least one part of the user's body.

In some embodiments, the body feature point information described above may be information of a joint point of the body. Specifically, identifying information of a joint point of a body from the image information may be implemented in the following way: Using the image information as an input parameter of a preset model, running the preset model to obtain joint point information of a body in the image information. The preset model may be a machine-learning model, such as a neural network model. The machine-learning model cited herein may be referred as a first machine-learning model to be distinguished from another machine-learning model. The first machine-learning model may use training samples, such as a set of labeled image samples, to perform the training and learning. The first machine-learning model after the learning and training may be used in recognization on image information to perform the task of recognizing articulation point of human being in the image information. More particularly, the image information may be input to the first machine-learning model completing the training and learning to run the first machine-learning model to obtain information on articulation points. More particularly, the training principle of the first machine-learning model may be briefly described as follows: inputting image samples into a first machine-learning model to obtain an outputting result; calculating a loss function according to a label indicating that the outputting result is corresponding to the image sample; optimizing the parameters in the first machine-learning model according to the loss function, if the loss function does not meet the converging requirement, and repeating the above steps by keeping using other image samples in the set of image samples to train the optimized first machine-learning model till the loss function meets the converging requirement.

In some embodiments, “collecting body feature point information of the user from the image information” in the above step of S1001 may be implemented by the following steps:

S1011. Performing identifying on the image information to identify joint points of the body of the user.

According to the description above, the step of S1011 may be specifically as follows: inputting the image information to the first machine-learning model completing the training and learning, and running the first machine-learning model to obtain the information on articulation points of human being.

S1012. Obtaining position information of the joint points of the body.

S1013. Using the position information of the joint point of the body as the body feature point information.

In some embodiments of the present disclosure, the collected image information containing the user's image may be three-dimensional information, and the position information of the identified joint point of the body may be a three-dimensional coordinate information of the joint point of the body of the user in the image information containing the user's image.

In some embodiments, the collected image information containing the user's image may be two-dimensional information, and the position information of the identified joint point of the body may be two-dimensional coordinate information of the joint point of the body in the image information containing the user's image.

Furthermore, the method provided in this embodiment further includes:

S1021, Obtaining an initial training model corresponding to a decomposed action in a training project.

S1022, Obtaining a set of samples.

S1023, Performing training of the initial training model by using the set of samples to obtain the action model.

In some embodiments of the present disclosure, different decomposed actions may correspond to different action models.

For example, the initial training model is a second machine-learning model, such as neural network learning model, e.g., convolutional neural network model, fully connected neural network or the like. The first machine-learning model cited above and the second machine-learning model cited herein may be two different models, and may have different neural network architectures. The first machine-learning model may be used in recognization on articulation point of human being in an image, and the second machine-learning model may be used in action evaluation, and thus the two models may use the training samples of different data types. For example, the training samples required for the first machine-learning training model may include: image samples and labels corresponding to the image samples, such as the labels of articulation points in the image samples; the training samples required for the second machine-learning training model may include: character point samples of human being and labels corresponding to the character samples, such as the labels indicating whether or not the action is right.

In another implementable technical solution, the action model in this embodiment is standard information used for comparing information. Correspondingly, “performing evaluation on action information of at least one part of the user's body in the image information by using the action model to obtain an evaluation result” in the above step of S103 may further be implemented by the following steps:

S1031, Performing identifying on the image information to identify joint points of the body of the user.

S1032, Obtaining a relative positional relationship between joint points of the body.

S1033, Determining action information of at least one part of the user's body according to a relative positional relationship between the joint points of the body.

S1034, Comparing action information of at least one part of the user's body with standard action information of a corresponding part in the action model to obtain an evaluation result of the action information of the at least one part.

In some embodiments of the present disclosure, the relative positional relationship between the joint points of the body may be analyzed to determine the type of action performed by the user, which is related to the action information of a part of the body. Alternatively, the above action information may be a relative positional relationship between joint points of the body.

In some embodiments of the present disclosure, the above action model may include standard action information corresponding to different parts of the body. An evaluation result of the action information of at least one part of the body may be obtained by comparing the obtained action information of at least one part of the user's body with the standard action information of the corresponding part in the action model. The evaluation result may be the similarity between the action information of the at least one part of the body and the standard action information of the corresponding part in the action model when compared with the standard action information. Specifically, it may be represented by a score of similarity. The higher the score is, the closer to the standard action information the action information of at least one part of the user's body in the image information is.

In some embodiments of the present disclosure, the relative positional relationship between the joint points of the body may be a relative coordinate positional relationship between the joint points of the body. For example, the standard action information in the action model may be the relative coordinate positions of the joint points of waist, foot, head, and hand corresponding to the training actions that the user refers to. The acquired action information of at least one part of the user's body is a relative coordinate position of the joint point of waist, foot, head, and hand corresponding to the acquired action of the at least one part of the user's body.

In some embodiments of the present disclosure, the “acquiring an action model corresponding to training actions that the user refers to” in the above step of S102 may be implemented by the following steps:

S1041, Obtaining the playing position of a current teaching video.

S1042, Determining a training action that the user refers to according to the playing position.

S1043, Acquiring an action model corresponding to the training action from a local source or on internet.

Alternatively, the current teaching video is a video of a training action that the user refers to during exercise, and the playing position of the current teaching video may be a playing timing corresponding to a frame corresponding to a training action that the user currently refers to in a total duration of the teaching video, or a number of a currently playing frame.

Alternatively, the “acquiring an action model corresponding to training actions that the user refers to” in the above step of S102 may also be implemented in the following steps:

S1051, Acquiring an action model corresponding to the training action matched with a learner level of which the user is according to the learner level; or

S1052, Obtaining an action model corresponding to the training action matched with the learner level selected by the user, in response to the selecting on the learner lever by the user.

In some embodiments of the present disclosure, users of different levels may also correspond to different accuracy in matching with the action model, and the action model may be divided into different levels according to the accuracy, such as low (L), Middle (M), and high (H). For a beginner, an action model of low accuracy may be used for matching, so that participants may have confidence and motivation to continue learning. For those who are continuously promoted, an action model of medium accuracy or further high accuracy may be used for matching, so that participants continue to have improvement and satisfaction.

Alternatively, the levels of the users may correspond to the levels of the action models on a one-to-one basis.

For example, the user's levels may be divided into primary, intermediate, and advanced, and the accuracy of the corresponding action model may be: low accuracy, medium accuracy, and high accuracy.

In some embodiments of the present disclosure, when the action model is used to evaluate action information of at least one part of the user's body in the image information to obtain an evaluation result, the levels of the users may be different, and the levels of the action models may be different, and thus corresponding different evaluation thresholds may be set to perform evaluation on the action information of at least one part of the user's body.

Alternatively, when the action information of at least one part of the user's body is compared with standard action information of a corresponding part in the action model to obtain an evaluation result of the action information of the at least one part, when the similarity between the action information of at least one part of the user's body and the standard action information is in different ranges, the obtained evaluation result is different.

For example, when the similarity between the action information of at least one part of the user's body and the standard action information is greater than 80% and less than 85%, the score corresponding to the evaluation result may be 80. When the similarity is greater than 85% and less than 90%, the score corresponding to the evaluation result may be 85.

Alternatively, the similarity range corresponding to the action models of different accuracy and the corresponding evaluation results may be different.

Furthermore, the accuracy level of each action model may be divided into different levels, such as L1, L2, L3, M1, M2, M3, H1, H2, and H3. Such level may be selected by the user or set by the system according to an algorithm.

In some embodiments of the present disclosure, the user may make registration in related APPs and then select a fitness course. Users may be divided into different levels and enjoy different free courses and other value-added services, such as free courses in a certain range, remotely calling for real-time coaching from a real trainer, etc. according to different fees, service life, and other factors.

Alternatively, the user may make registration and exercise course selection by touching one or more of mobile phone or tablet app, MIC voice, infrared or Bluetooth remote control, and keyboard and mouse. The touching on the mobile phone or tablet app is preferred.

In some embodiments of the present disclosure, the evaluation method may collect human gesture control actions through a camera, and calculate and identify the intention of the actions, so as to control the display for providing a reference video for the user or for playing the user's action playback video. For example, the user may wave palm from top to bottom to switch the display on the screen.

Alternatively, if the user selects a course that is not in the range of free (a course that requires payment), the fee can be paid with a mobile phone or tablet app. There are two methods for mobile or tablet App payment, payment by scanning or online. The payment by scanning may be in the following scenario: after a user selects a course on the display by touching, if the course requires payment, the user may scan a QR code for the course so as to make a payment with a mobile phone or tablet app. The online payment may be in the following scenario: a user selects a course with a mobile phone or tablet app, and if the course requires payment, the user may make the payment for the course online directly with the mobile phone or tablet app.

In some embodiments, the method may further include:

S1061, Obtaining training information related to the user in response to a calling instruction initiated by the user.

S1062, Determining information of a corresponding trainer based on the training information.

S1063, Sending a calling request to a terminal used by the trainer according to the information of the trainer.

In some embodiments of the present disclosure, the training information may include at least one of the following: user's level information, user's evaluation result, and user's historical exercise information.

In some embodiments, the trainer information described above may include at least one of the following: working experience of a trainer, a fee schedule for the teaching by the trainer, gender information of a trainer, age information of a trainer, a expertise field of a trainer, and contact information of a trainer.

Specifically, the corresponding trainer information may be determined based on the training information as follows:

Comparing the training information with a plurality sets of matching condition information associated with pre-stored trainer identifiers to obtain similarity between the training information and each set of matching condition information;

Using the information corresponding to the trainer identifier associated with the matching condition information whose similarity satisfies a preset condition as the trainer information. The trainer identifier may be a user ID of a trainer, and the matching condition information may be level information of an user with interest input by the trainer himself, the evaluation result of the user with interest, and the historical exercise information of the user with interest. The preset condition may be that similarity is greater than a preset value. A trainer identifier may correspond to a set of matching condition information.

In some embodiments, the trainer information corresponding to a new user and the user with fitness experience may be different, the trainer information corresponding to different levels of users may be different, and the trainer information corresponding to different evaluation results may be different.

In some embodiments, the method further includes:

S1071, Estimating the amount of exercise of the at least one part of the user's body according to the action information of the at least one part of the user' body to obtain a first estimation result.

S1072, Highlighting a corresponding part on the image information according to the first estimation result.

For example, estimation may be made on the amount of exercise on the muscles of the user's body and a body heat map may be displayed (muscles under large amount of exercise are displayed in red, the larger the amount of exercise is, the darker the red is). Specifically, estimation may be made on the amount of exercise on the biceps brachii muscle of the user, and a heat map corresponding to the biceps brachii muscle may be displayed.

In some embodiments, estimation may be also made on the amount of exercise on the muscles of the whole body of the user, and a heat map corresponding to the whole body may be displayed.

In some embodiments, during the exercise, estimation may be made on the amount of user's exercise based on the action information of at least one part of the user's body and the corresponding exercise duration.

In some embodiments, the method further includes:

S1081, Acquiring first characteristic information of a user, and the first characteristic information includes height information and/or weight information.

S1082, Performing estimation on the consumption of calorie consumed by the user during the exercise by using the first feature information and the user's exercise duration to obtain a second estimation result.

S1083, Outputting the second estimation result.

In some embodiments, the first feature information of the user may be input by the user, or may be automatically obtained by performing identifying based on the captured user image information. Specifically, the height information of the user may be automatically determined based on the size information of the image in the captured user image information and the height information of the user in the image information, and the user's weight information may be determined based on the size information of the image and the area occupied by the user in the image when the user is standing.

In some embodiments, the first feature information of the user may be detected by setting a sensor at the scene where the user exercises.

In some embodiments, the method further includes:

S1091, Generating and outputting encouragement information, if the evaluation score corresponding to the evaluation result is greater than a first preset threshold, and generating and outputting error warning information, if the evaluation score corresponding to the evaluation result is less than a second preset threshold.

In some embodiments, the evaluation result may be score information. The first preset threshold and the second preset threshold may be set by a user, may be automatically generated by the system according to the user's level, and may be set in advance by a trainer.

In some embodiments of the present disclosure, the evaluation result described above may be information used to evaluate whether the action information of at least one part of the user is correct.

In some embodiments of the present disclosure, a total evaluation result corresponding to the action information of a plurality of parts that the action is right may be generated, when the ratio of the number of parts corresponding to the evaluation result identifying the action is right to the number of parts corresponding to the evaluation result identifying the action is wrong is greater than a preset ratio, when the action information of a plurality of parts is under evaluation at the same time.

In some embodiments of the present disclosure, the above evaluation result, encouragement information, and error warning information may be displayed in a reference video corresponding to a training action that the user currently refers to, and may specifically be displayed at a part of the body of the character corresponding to at least one part of the user in the reference video.

In some embodiments, the method further includes:

S11, Acquiring a user's playing instruction.

S12, Playing a media file within a preset historical time period including at least one of the following content according to the playing instruction: the image information, the evaluation result, the error warning information, and the encouragement information.

In some embodiments of the present disclosure, the playing instruction of the user is an instruction of the user to playback his own action video. During playback, the error warning information may be shown in a form of image or text on the screen or in a form of voice through the speaker.

In some embodiments, the method further includes:

S13, Generating an exercise report, including: the user's exercise duration, the first estimation result, the second estimation result, the evaluation result, the error warning information, and the encouragement information.

In some embodiments, the method further includes:

S14, Acquiring a user's sharing instruction.

S15, Sending the exercise report to a preset terminal according to the sharing instruction.

In some embodiments of the present disclosure, the user may share the exercise report to a social software, and the preset terminal may be a terminal used by the user himself, or may be a terminal of a corresponding trainer.

In some embodiments, the method further includes:

S16, Acquiring gesture information of a user.

S17, Analyzing the gesture information to obtain a control instruction corresponding to the gesture information.

S18, Executing the control instruction.

In some embodiments of the present disclosure, the above gesture information may be an instruction for controlling a screen to display a video corresponding to a training action that a user refers to, for example:

In some embodiments, the gesture information may be a palm waving from top to bottom, and the corresponding control instruction may be an instruction to control the screen to switch the display.

In some embodiments, the method further includes:

S001, Acquiring second characteristic information of a user, including at least one of the following: heart rate information of the user, and breathing frequency of the user.

S002, Generating alarm information when a value corresponding to the second characteristic information exceeds a corresponding preset range of value.

S003, Outputting the alarm information.

In some embodiments of the present disclosure, the user may wear a sensor for measuring heart rate information and/or breathing frequency. In this embodiment, the user's heart rate information and the user's breathing frequency may be obtained by the information sent by the sensor. When the user's heart rate information and the user's breathing frequency exceed the normal range of heart rate information and/or breathing frequency corresponding to a healthy person, an alarm message may be generated. Specifically, the alarm message may be output in at least one of the following ways: video outputting, text outputting. The sensor worn by the user may be a watch, a bracelet, or other devices.

The technical solution provided in the present disclosure may be used in all kinds of scenarios requiring action teaching, such as fitness, dance, rehabilitation, industry posture training and teaching. Taking fitness training as an example, this solution may place the apparatus corresponding to this solution in the gym, even the novel unattended gym, or the user's home or office, etc., which is convenient for users to obtain real-time, professional and private trainer at any time with low cost. users time may be saved and users' fitness costs may be saved.

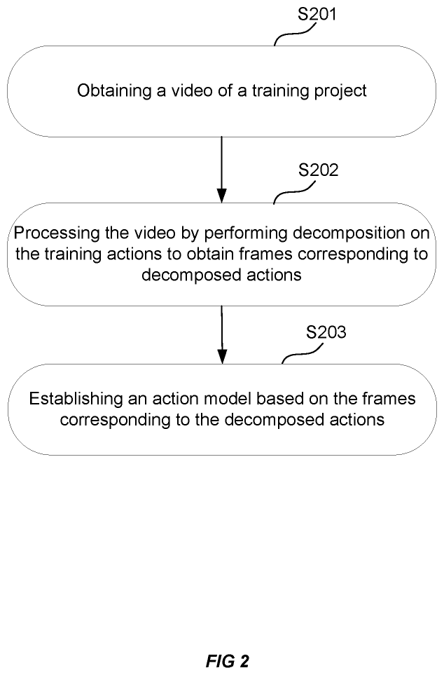

FIG. 2 is a schematic flowchart of a model establishing method according to an embodiment of the present disclosure. The execution subject of the method provided by the embodiments of the present disclosure may be a device, which may be, but is not limited to, a device incorporated in any terminals, such as a smartphone, a tablet computer, a PDA (Personal Digital Assistant), a smart TV, a laptop, a portable computer, desktop computer, and smart wearable device. As shown in FIG. 2, the model establishing method includes:

S201, Obtaining a video of a training project;

S202, Processing the video by performing decomposition on the training actions to obtain frames corresponding to decomposed actions;

S203, Establishing an action model based on the frames corresponding to the decomposed actions.

The action model is used to perform evaluation on action information of at least one part of a user's body in collected image information containing a user's image.

In some embodiments of this disclosure, the action model herein may be same as the action model in S103 in the embodiment corresponding to FIG. 1.

In some embodiments, the method further includes:

S2001, Obtaining a set of samples.

S2002, Using the set of samples to train the action model to optimize parameters in the action model.

In some embodiments of the present disclosure, the set of samples may be derived from a teaching video of a trainer.

In some embodiments, each set of samples may correspond to one part of body, that is to say, one part of body may correspond to one action model. Each set of samples may correspond to a plurality of parts of body, that is to say, a plurality of parts of body may correspond to one action model.

In another technical solution, the action model may be standard information used for information comparison. Correspondingly, “Establishing an action model based on the frames corresponding to the decomposed actions” in the above step of S203 may be implemented by the following steps:

S2011, Performing identifying of a part of body on a frame to obtain an identifying result.

S2012. Establishing sub-models corresponding to a plurality of parts of the body based on the identifying result.

S2013, Obtaining the action model based on the sub-models corresponding to the plurality of parts of the body.

In some embodiments of the present disclosure, the frame herein may be a frame corresponding to the action of the trainer, different parts of body may correspond to different sub-models, and a plurality of sub-models constitute an action model.

In other embodiments of the present disclosure, the sub-model herein may be same as the action model in S103 in the embodiment corresponding to FIG. 1.

In some embodiments, the method further includes: storing a video of the training project in association with the action model.

In some embodiments, a video of a training project corresponds to a set of action models. For example, when the video of a training project is Tai Chi, the Tai Chi video corresponds to a set of action models, and a frame corresponding to each decomposed action of Tai Chi corresponds to one action model. For example: the frame corresponding to an action “starting up” in Tai Chi corresponds to action model 1, and the frame corresponding to the action “white crane bright wings” in Tai Chi corresponds to action model 2.

The operating principle and process of the embodiment corresponding to FIG. 2 may refer to the foregoing embodiment corresponding to FIG. 1, and details are omitted herein to avoid redundancy.

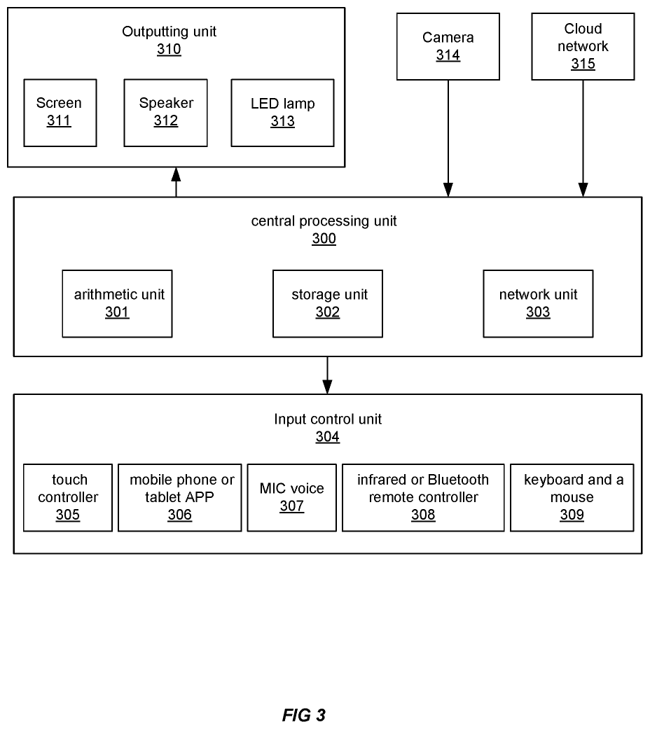

FIG. 3 shows a schematic structural diagram of a teaching system provided by an embodiment of the present disclosure. The components of the teaching system are shown in FIG. 3. The teaching system includes a central processing unit 300, an input control unit 304, an output unit 310, a camera. 314, and a cloud network 315.

The output unit 310 includes a screen 311, a speaker 312, and an LED lamp 313. The central processing unit 300 includes: an arithmetic unit 301, a storage unit 302, and a network unit 303. The input control unit 304 includes a touch controller 305, a mobile phone or tablet App 306, a MIC voice 307, an infrared or Bluetooth remote controller 308, a keyboard and a mouse 309.

Table 1 is the component classification of components of the above teaching system, and the optional and required situation of each component.

TABLE 1
The classification of the components of the teaching
system and the optional and necessity.

| Component | Component | Component | Required/ | |
|---|---|---|---|---|
| number | name | classification | optional | Description |
| 300 | central | Major class | required | / |
| | processing unit | | | |
| 301 | arithmetic unit | Subclass | Required | / |
| 302 | storage unit | Subclass | Required | / |
| 303 | network element | Subclass | Required | / |
| 304 | input control unit | Major class | Required | Preferably |
| | | | | 305 or 306 |
| 305 | touch controller | Subclass | Optional | / |
| 306 | mobile or tablet | Subclass | Optional | / |
| | app | | | |
| 307 | MIC voice | Subclass | Optional | / |
| 308 | IR or Bluetooth | Subclass | Optional | / |
| | remote controller | | | |
| 309 | keyboard and | Subclass | Optional | / |
| | mouse | | | |
| 310 | output unit | Major class | Required | / |
| 311 | Display | Subclass | Required | / |
| 312 | horn | Subclass | Required | / |
| 313 | LED lights | Subclass | Optional | / |
| 314 | camera | Major class | Required | Sometimes |
| | | | | also used |
| | | | | as a gesture |
| | | | | control |
| | | | | in 304 |
| 315 | cloud network | Major class | Optional | / |

The input control unit 304, the output unit 310, and the camera 314 in the teaching system are connected to the central processing unit 300 by an electrical connection or a wireless network. The cloud network 315 is connected to the central processing unit 300 by a wired or wireless network.

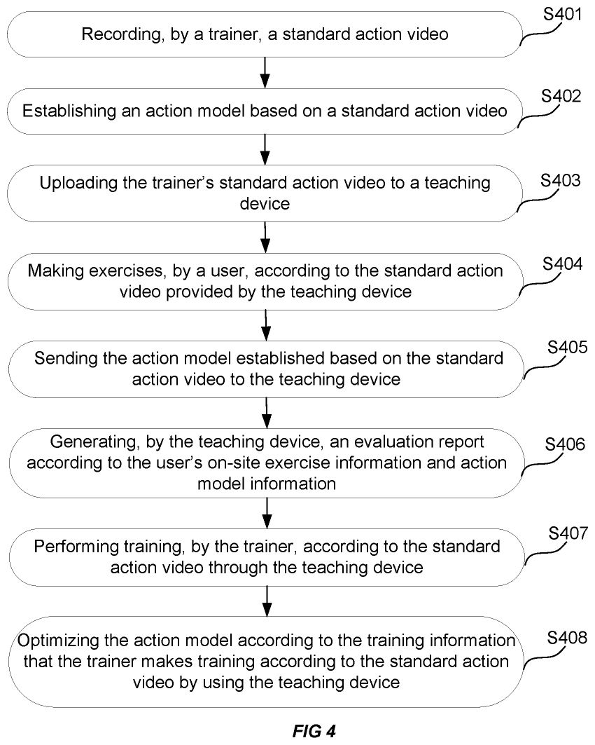

FIG. 4 is a schematic flowchart of an evaluation method according to an embodiment of the present disclosure.

The method includes the following steps:

S401, Recording, by a trainer, a standard action video.

S402, Establishing an action model based on a standard action video.

S403, Uploading the trainer's standard action video to a teaching device.

S404, Making exercises, by a user, according to the standard action video provided by the teaching device.

S405, Sending the action model established based on the standard action video to the teaching device.

S406, Generating, by the teaching device, an evaluation report according to the user's on-site exercise information and action model information. The evaluation report includes exercise suggestions, and recommendation of exercise type or trainer.

S407, Performing training, by the trainer, according to the standard action video through the teaching device;

S408, Optimizing the action model according to the training information that the trainer makes training according to the standard action video by using the teaching device.

The present disclosure further provides an evaluation method, which can be implemented in the following ways:

Recording a set of standard action videos of a trainer for a fitness course, such as yoga. Decomposing actions and marking the actions on the videos with respect to the main points and videos of fitness actions. Establishing model with respect to each decomposed action to form the initial yoga action model. Recording a set of standard action videos of a trainer for another fitness course, such as Tai Chi, and form the initial action model of Tai Chi.

In some embodiments, the standard action videos of a trainer for different fitness courses and their action models may constitute a trainer standard action video database (referred as a video database) and an action model database (referred as a model database), respectively. Video database and model database may be collectively referred as the database. For the off-line version, the database may be stored in the storage unit 302 in FIG. 3. For the network version, part or all of the database may be stored in the cloud network 315 in FIG. 3, or in the storage unit 302.

In some embodiments, artificial intelligence and deep learning may be adopted, so that the trainer himself may use the initial action model a lot of times and perform training repeatedly, and the action model database may become more intelligent and more versatile.

In some embodiments, the student (or user) makes registration first, and then selects a fitness course through the input control unit 304 in FIG. 3. Users may be classified into different levels and enjoy different free courses and other value-added services, such as free courses in a certain range, remotely calling real-time teaching by a real trainer, etc. according to different fees, service life, and other factors.

In some embodiments, the user controls the teaching device through the input control unit 304 in FIG. 3, and the control method may be one or more of the touch controller 305, the mobile phone or tablet App 306, the MIC voice 307, the infrared or Bluetooth remote controller 308, and the keyboard and mouse 309 in FIG. 3. The touch controller 305, mobile phone or tablet App 306 are preferable.

In some embodiments of the present disclosure, human gesture control actions may be collected through the camera 314 in FIG. 3, and the central processing unit 300 in FIG. 3 may be used to calculate and identify the intention of the actions, so as to control the teaching device. For example, the user may wave palm from top to bottom to switch the display on the screen 311 in FIG. 3, so as to implement the control on the teaching device by the input control unit 304 in FIG. 3.

Alternatively, if the user selects a course that is not in the range of free (a course that requires payment), the fee can be paid with a mobile phone or tablet app 306 in FIG. 3. There are two payment methods for mobile or tablet App 306 in FIG. 3, payment by scanning or online. The payment by scanning may be in the following scenario: after a user selects a course on the screen 311 in FIG. 3 by using the touch controller 305 in FIG. 3, if the course requires payment, the user may scan a QR code for the course so as to make a payment by using mobile or tablet App 306 in FIG. 3. The online payment may be in the following scenario: a user selects a course with by using mobile or tablet App 306 in FIG. 3, and if the course requires payment, the user may make the payment for the course online directly with by using mobile or tablet App 306 in FIG. 3.

In some embodiments, after the user selects and starts the course, the user makes exercises by watching the video on the screen 311 in FIG. 3 in the teaching device, and the camera 314 in FIG. 3 collects body actions, the central processing unit 300 in FIG. 3 may perform operations and identifying actions, and compare the actions with the model database.

In some embodiments, when the user action is compared with the action in the action model database, the comparison may be made in different levels according to the matching accuracy, such as low (L), Middle (M), and high (H). For a beginner, an action model of low accuracy may be used for matching, so that participants may have confidence and motivation to continue learning. For those who are continuously promoted, an action model of medium accuracy or further high accuracy may be used for matching, so that participants continue to have improvement and satisfaction. The levels of accuracy for matching may be determined by the program using different thresholds when identifying actions. Furthermore, each level may be divided into sub-levels, such as nine sub-levels of L1, L2, L3, M1, M2, M3, H1, H2, and H3. Such level may be selected by the user or set by the system according to the algorithm.

According to the results of the comparison between the user's actions and the actions of the action model database, the central processing unit 300 in FIG. 3 may display the results of the comparison between the user's actions and the actions of the action model database by using the screen 311 in FIG. 3 to display images and text, or use the speaker 312 in FIG. 3 to prompt in voice. For example, when the user is doing right actions, encouragement images, text, and voice may be output; when the user is doing wrong actions, the position where the user is doing wrong may be marked with images on the trainer standard action video, the error message may be displayed for explanation by text, and further prompt the error by voice.

In some embodiments, after learning a set of courses, the user may play back and review the wrong action information during the exercise. During playback, the wrong action information may be displayed in a form of images or text on the screen 311 in FIG. 3, or output in a form of voice through the speaker 312 in FIG. 3.

In some embodiments, the user may call a real trainer to perform remote real-time teaching during a practice or during playback.

In some embodiments, after a set of actions of the course is completed, the central processing unit 300 in FIG. 3 may generate an exercise report and a QR code, and display them on the screen 311 in FIG. 3. The user may scan the QR code by using Mobile phone or tablet App 306 in FIG. 3 for social sharing.

In some embodiments, during the exercise, the teaching device may perform estimation on the amount of exercise of the muscles of the user's body by using the user's action information collected by the camera 314 in FIG. 3, and display a body heat map (muscles are displayed in red with large amounts of exercise, the greater the amount of exercise is, the darker the red is) in real time on the screen 311 in FIG. 3.

In some embodiments, the exercise report includes the matching degree of the user action collected by the camera with the actions in the standard action database, the intensity of the user's exercise, the duration of the user's exercise, the estimation of the user's calorie consumption, and the like.

In some embodiments, the user may input the height and weight parameters before exercise to make the calorie consumption estimated by the trainer more accurate.

In some embodiments, the user may wear a watch, bracelet, or other device with a heart rate measurement function, which may be wirelessly connected to the teaching device and transmit the heart rate information obtained by measurement to the teaching device. The teaching device may monitor the heart rate information of the user during exercise, and output warnings and suggestions when it is too high. Similar monitoring may be made on breathing can be monitored similarly.

In some embodiments, a trainer or a fitness training institution (referred as a third party) may register as a supplier, record a course on the teaching device, provide key points of actions and action decomposition, and save it as a third party course on cloud network 315 in FIG. 3. When a user chooses a course of the third-party, some of the payment can be offered to the third party.

The teaching device of the present disclosure may be used in all scenarios requiring action teaching such as fitness, dance, rehabilitation, industry posture training and teaching. Taking the fitness disclosure as an example, the teaching device may be placed in a gym, even the novel unattended gym, or the user's home or office, etc., which is convenient for users to obtain professional personal teaching at any time and at low cost.

The present solution may realize real-time high-precision instruction of action teaching. The screen 311 in FIG. 3 may be mirror glass without opening on the outer surface. The camera 314 in FIG. 3 may be set as a hidden camera in the mirror glass. When no power is supplied to the teaching device, the screen is a standard mirror when viewed from the front. After the teaching device is powered on, it is an action teaching device with a screen.

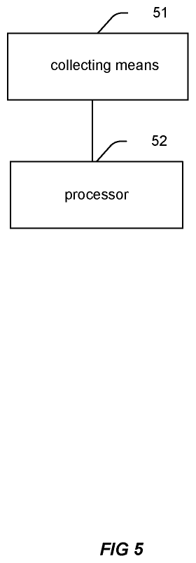

FIG. 5 shows a teaching device provided by an embodiment of the present disclosure. The teaching device includes: a collecting means 51 configured to collect image information including a user's image; a processor 52 configured to acquire an action model corresponding to training actions that the user refers to, and performing evaluation on action information of at least one part of the user's body in the image information by using the action model to obtain an evaluation result, output the evaluation result to an outputting means.

The collecting means 51 may be same as the camera 314 in FIG. 3, and the processor 52 may be same as the central processing unit 300 in FIG. 3.

The operating principle and process of the embodiment corresponding to FIG. 5 may refer to the foregoing embodiment corresponding to FIGS. 1 and 3, and details are omitted herein to avoid redundancy.

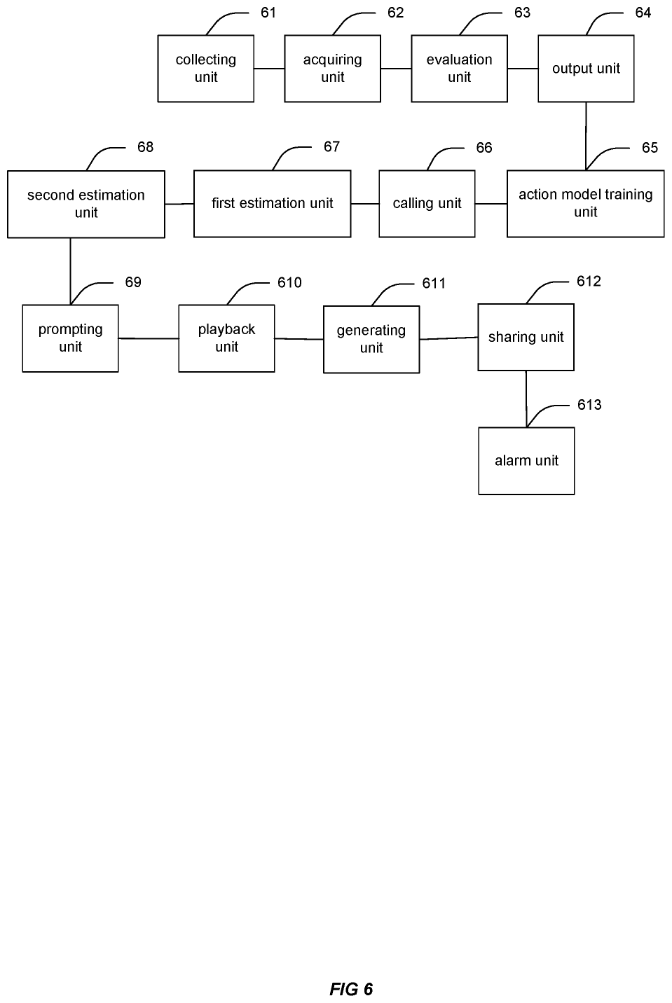

FIG. 6 shows an evaluation device provided by an embodiment of the present disclosure. As shown in FIG. 6, the device includes: a collecting unit 61 configured to collect image information including a user's image; an acquiring unit 62 configured to acquire an action model corresponding to training actions that the user refers to; an evaluation unit 63 configured to use the action model to perform evaluation on action information of at least one part of the user's body in the image information, to obtain an evaluation result; an output unit 64 configured to output an evaluation result of action information of at least one part of the user's body.

Alternatively, the evaluation unit 63 configured to use the action model to perform evaluation on action information of at least one part of the user's body in the image information, to obtain an evaluation result, is specifically configured to: collect body feature point information of the user from the image information; use the body feature point information as an input parameter of the action model, run the action model, and obtain an evaluation result of action information of at least one part of the user's body.

Alternatively, the evaluation unit 63 configured to use the action model to perform evaluation on action information of at least one part of the user's body in the image information, to obtain an evaluation result, is specifically configured to perform identifying on the image information to identify joint points of the body of the user; obtain position information of the joint points of the body; use the position information of the joint point of the body as the body feature point information.

In some embodiments, the device further includes an action model training unit 65 configured to: obtain an initial training model corresponding to a decomposed action in a training project; obtain a set of samples; perform training of the initial training model by using the set of samples to obtain the action model.

In some embodiments, the evaluation unit 63 configured to use the action model to perform evaluation on action information of at least one part of the user's body in the image information, to obtain an evaluation result, is specifically configured to perform identifying on the image information to identify joint points of the body of the user; obtaining a relative positional relationship between joint points of the body; determine action information of at least one part of the user's body according to a relative positional relationship between the joint points of the body; compare action information of at least one part of the user's body with standard action information of a corresponding part in the action model to obtain an evaluation result of the action information of the at least one part.

In some embodiments, an acquiring unit 62 configured to acquire an action model corresponding to training actions that the user refers to, is specifically configured to: obtain the playing position of a current teaching video; determine a training action that the user refers to according to the playing position; acquire an action model corresponding to the training action from a local source or on internet.

In some embodiments, an acquiring unit 62 configured to acquire an action model corresponding to training actions that the user refers to, is specifically configured to: acquire an action model corresponding to the training action matched with a learner level of which the user is according to the learner level; or obtain an action model corresponding to the training action matched with the learner level selected by the user, in response to the selecting on the learner lever by the user.

In some embodiments, the device further includes a calling unit 66, configured to obtain training information related to the user in response to a calling instruction initiated by the user; determine information of a corresponding trainer based on the training information; send a calling request to a terminal used by the trainer according to the information of the trainer.

In some embodiments, the device further includes a first estimation unit 67, configured to: estimate the amount of exercise of the at least one part of the user's body according to the action information of the at least one part of the user' body to obtain a first estimation result; highlight a corresponding part on the image information according to the first estimation result.

In some embodiments, the device further includes a second estimation unit 68 configured to acquire first characteristic information of a user, and the first characteristic information includes height information and/or weight information; perform estimation on the consumption of calorie consumed by the user during the exercise by using the first feature information and the user's exercise duration to obtain a second estimation result; output the second estimation result.

In some embodiments, the device further includes a prompting unit 69, configured to: generate and output encouragement information, if the evaluation score corresponding to the evaluation result is greater than a first preset threshold, and generate and output error warning information if the evaluation score corresponding to the evaluation result is less than a second preset threshold.

In some embodiments, the device further includes: a playback unit 610, configured to: acquire a user's playing instruction; play a media file within a preset historical time period including at least one of the following content according to the playing instruction: the image information, the evaluation result, the error warning information, and the encouragement information.

In some embodiments, the device further includes a generating unit 611, configured to generate an exercise report, including: the user's exercise duration, the first estimation result, the second estimation result, the evaluation result, the error warning information, and the encouragement information.

In some embodiments, the device further includes a sharing unit 612, configured to: acquire a user's sharing instruction; send the exercise report to a preset terminal according to the sharing instruction.

In some embodiments, the device is further configured to: acquire gesture information of a user; analyze the gesture information to obtain a control instruction corresponding to the gesture information; execute the control instruction.

In some embodiments, the device further includes an alarm unit 613, configured to acquire second characteristic information of a user, including at least one of the following: heart rate information of the user, and breathing frequency of the user; generate alarm information when a value corresponding to the second characteristic information exceeds a corresponding preset range of value; output the alarm information.

The operating principle and process of each module of the evaluation device provided by FIG. 6 in the embodiment of the present disclosure may refer to the evaluation method of foregoing embodiment in FIG. 1, and details are omitted herein to avoid redundancy.

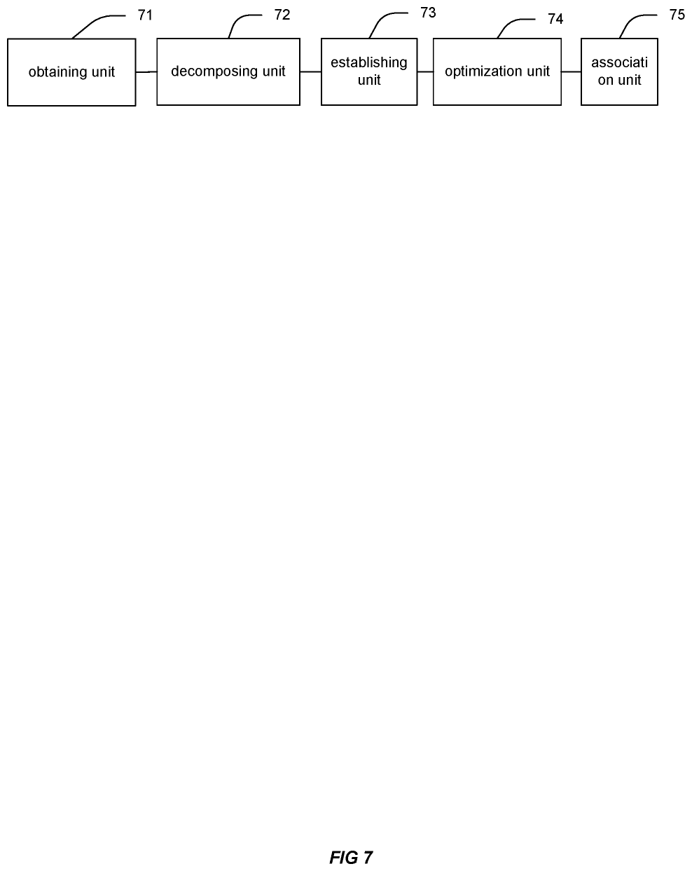

FIG. 7 illustrates a model establishing device provided by an embodiment of the present disclosure. As shown in FIG. 7, the device includes: an obtaining unit 71 configured to obtain a video of a training project; a decomposing unit 72 configured to process the video by performing decomposition on the training actions to obtain frames corresponding to decomposed actions; an establishing unit 73 configured to establish an action model based on the frames corresponding to the decomposed actions.

The action model is used to perform evaluation on action information of at least one part of a user's body in collected image information containing a user's image.

In some embodiments, the device further includes an optimization unit 74, configured to: obtain a set of samples; use the set of samples to train the action model to optimize parameters in the action model.

In some embodiments, the establishing unit 73 configured to establish an action model based on the frames corresponding to the decomposed actions, is specifically configured to: perform identifying of a part of body on a frame to obtain an identifying result; establish sub-models corresponding to a plurality of parts of the body based on the identifying result; obtain the action model based on the sub-models corresponding to the plurality of parts of the body.

In some embodiments, the device further includes an association unit 75, configured to store a video of the training project in association with the action model.

The operating principle and process of each module of the model establishing device provided by FIG. 7 in the embodiment of the present disclosure may refer to the model establishing method of foregoing embodiment in FIG. 2, and details are omitted herein to avoid redundancy.

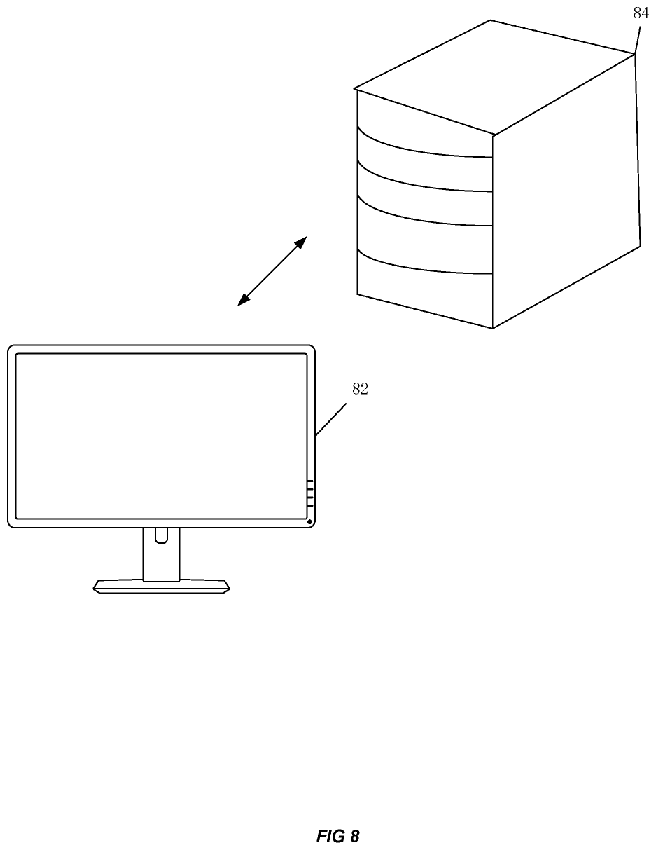

FIG. 8 illustrates a teaching system provided by an embodiment of the present disclosure, including:

A teaching device 82 configured to collect image information containing a user's images; and send the image information to a server.

A server 84 configured to acquire an action model corresponding to training actions that the user refers to; perform evaluation on action information of at least one part of the user's body in the image information by using the action model to obtain an evaluation result; sending the evaluation result to the teaching device.

The teaching device is further configured to output the evaluation result on the action information of at least one part of the user's body.

The operating principle and process of the teaching system provided by FIG. 8 in the embodiment of the present disclosure may refer to the evaluation method of foregoing embodiment in FIG. 1, and details are omitted herein to avoid redundancy.

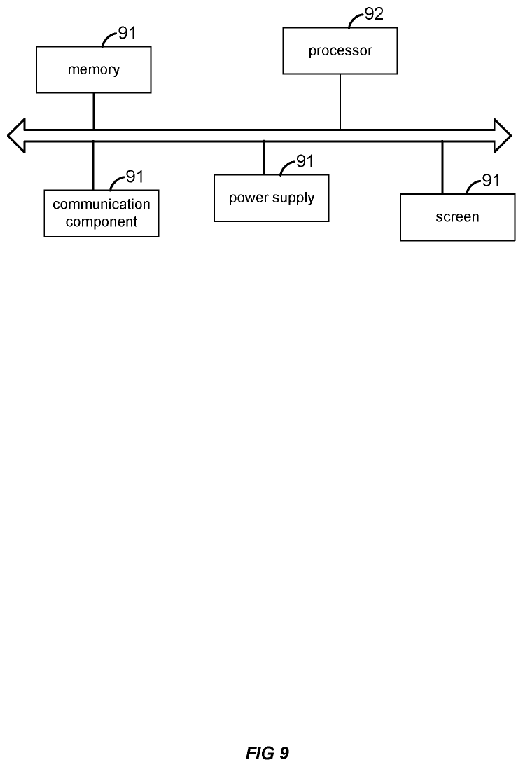

FIG. 9 is a schematic structural diagram of an electrical apparatus according to an embodiment of the present disclosure. As shown in FIG. 9, the electrical apparatus includes: a memory 91 and a processor 92.

The memory 91 is configured to store a program.

The processor 92 is coupled to the memory, and is configured to execute the program stored in the memory, to: collect image information containing a user's image; acquire an action model corresponding to training actions that the user refers to; perform evaluation on action information of at least one part of the user's body in the image information by using the action model to obtain an evaluation result; output the evaluation result on the action information of at least one part of the user's body. The memory 91 described above may be configured to store various other data to support operations on a computing device. Examples of such data include instructions of any APP or method running on a computing device. The memory 91 may be implemented by any type of volatile or non-volatile storage device or a combination thereof, such as static random access memory (SRAM), electrically erasable programmable read-only memory (EEPROM), Programming read-only memory (EPROM), programmable read-only memory (PROM), read-only memory (ROM), magnetic memory, flash memory, magnetic disk or optical disk.

In addition to the above functions, the processor 92 configured to execute the program stored in the memory 91, may implement other functions. Details may refer to the descriptions of the foregoing embodiments.

Further, as shown in FIG. 9, the electrical apparatus further includes: a display 93, a power supply 94, a communication component 95 and other components. Only some of the components are shown schematically in FIG. 9, which does not mean that the electrical apparatus includes only the components shown in FIG. 9.

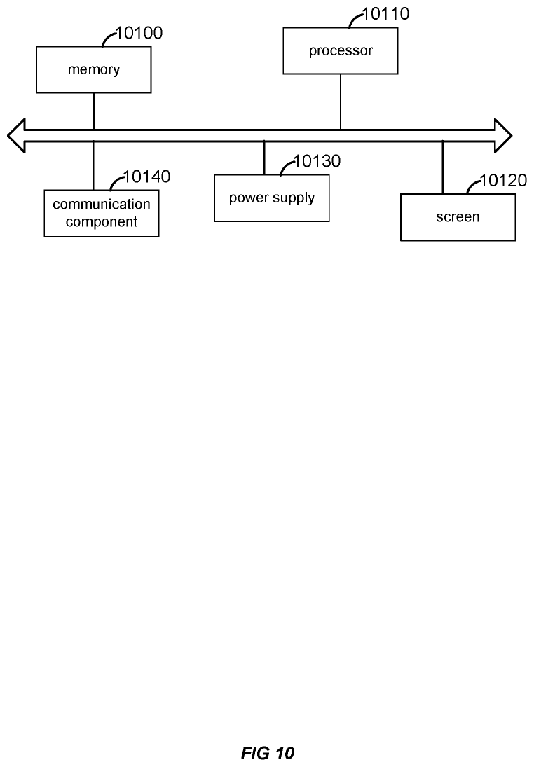

FIG. 10 is a schematic structural diagram of an electrical apparatus according to an embodiment of the present disclosure. As shown in FIG. 10, the electrical apparatus includes: a memory 10100 and a processor 10110.

The memory 10100 is configured to store a program.

The processor 10110 is coupled to the memory, and is configured to execute the program stored in the memory, to: obtain a video of a training project; process the video by performing decomposition on the training actions to obtain frames corresponding to decomposed actions; establish an action model based on the frames corresponding to the decomposed actions, wherein the action model is used to perform evaluation on action information of at least one part of a user's body in collected image information containing a user's image.

The memory 10100 described above may be configured to store various other data to support operations on a computing device. Examples of such data include instructions for any APP or method operating on a computing device. The memory 10100 may be implemented by any type of volatile or non-volatile storage devices or a combination thereof, such as static random access memory (SRAM), electrically erasable programmable read-only memory (EEPROM), Programming read-only memory (EPROM), programmable read-only memory (PROM), read-only memory (ROM), magnetic memory, flash memory, magnetic disk or optical disk.

In addition to the above functions, the processor 10110 configured to execute the program stored in the memory 10100, may implement other functions. Details may refer to the descriptions of the foregoing embodiments.

Further, as shown in FIG. 10, the electrical apparatus further includes: a display 10120, a power supply 10130, a communication component 10140 and other components. Only some of the components are shown schematically in FIG. 10, which does not mean that the electrical apparatus includes only the components shown in FIG. 10.

Correspondingly, an embodiment of the present disclosure further provides a computer-readable storage medium storing a computer program, which when executed by a computer can implement the steps or functions of the evaluation methods provided by the foregoing embodiments.

Correspondingly, the embodiment of the present disclosure further provides a computer-readable storage medium storing a computer program, and the computer program, when executed by a computer, can implement the steps or functions of the model establishing method provided by the foregoing embodiments.

The device embodiments described above are only schematic, and the units described as separate components may or may not be physically separated, and the components shown as units may or may not be physical units, that is, may be located at a place, or may be distributed on network units. Some or all of the modules may be selected according to actual needs to achieve the objective of the solution of this embodiment. The skilled in the art may understand and implement without creative work.

With the description of the above embodiments, the skilled in the art can clearly understand that each embodiment can be implemented by means of software with a necessary universal hardware platform, and of course, also by hardware. Based on such an understanding, the above-mentioned technical solution essentially or part that contributes to the existing technology can be embodied in the form of a software product, which can be stored in a computer-readable storage medium, such as ROM/RAM, magnetic A disc, an optical disc, and the like including instructions for rendering a computer device (which may be a personal computer, a server, or a network device, etc.) to execute the methods described in various embodiments or certain parts of the embodiments.

Finally, it should be noted that the above embodiments are only used to describe the technical solution of the present disclosure, and are not limited thereto. Although the present disclosure has been described in detail with reference to the foregoing embodiments, the skilled in the art should understand that they can still modify the technical solutions described in the foregoing embodiments, or equivalently replace some of the technical features thereof. These modifications or replacements do not deviate the essence of the corresponding technical solutions from the spirit and scope of the technical solutions of the embodiments of the present disclosure.

1. An evaluation method, comprising:
collecting image information containing a user's image;
acquiring an action model corresponding to training actions that the user refers to;
performing evaluation on action information of at least one part of the user's body in the image information by using the action model to obtain an evaluation result;
outputting the evaluation result on the action information of at least one part of the user's body.

2. The method according to claim 1, wherein the performing evaluation on action information of at least one part of the user's body in the image information by using the action model to obtain an evaluation result comprises:
collecting body feature point information of the user from the image information;
using the body feature point information as an input parameter of the action model, running the action model, and obtaining an evaluation result of action information of at least one part of the user's body.

3. The method according to claim 2, wherein the collecting body feature point information of the user from the image information comprises:
performing identifying on the image information to identify joint points of the body of the user;
obtaining position information of the joint points of the body;
using the position information of the joint point of the body as the body feature point information.

4. The method according to claim 1, further comprising:
obtaining an initial training model corresponding to a decomposed action in a training project;
obtaining a set of samples;
performing training of the initial training model by using the set of samples to obtain the action model.

5. The method according to claim 1, wherein the performing evaluation on action information of at least one part of the user's body in the image information by using the action model to obtain an evaluation result comprises:
performing identifying on the image information to identify joint points of the body of the user;
obtaining a relative positional relationship between joint points of the body;
determining action information of at least one part of the user's body according to a relative positional relationship between the joint points of the body;
comparing action information of at least one part of the user's body with standard action information of a corresponding part in the action model to obtain an evaluation result of the action information of the at least one part.

6. The method according to claim 1, wherein the acquiring an action model corresponding to training actions that the user refers to comprises:
obtaining the playing position of a current teaching video;
determining a training action that the user refers to according to the playing position;
acquiring an action model corresponding to the training action from a local source or on internet.

7. The method according to claim 1, the acquiring an action model corresponding to training actions that the user refers to comprises:
acquiring an action model corresponding to the training action matched with a learner level of which the user is according to the learner level; or
obtaining an action model corresponding to the training action matched with the learner level selected by the user, in response to the selecting on the learner lever by the user.

8. The method according to claim 1, further comprising:
obtaining training information related to the user in response to a calling instruction initiated by the user;
determining information of a corresponding trainer based on the training information;
sending a calling request to a terminal used by the trainer according to the information of the trainer.

9. The method according to claim 1, further comprising:
estimating the amount of exercise of the at least one part of the user's body according to the action information of the at least one part of the user' body to obtain a first estimation result;
highlighting a corresponding part on the image information according to the first estimation result.

10. The method according to claim 9, further comprising:
acquiring first characteristic information of a user, and the first characteristic information comprises height information and/or weight information;
performing estimation on the consumption of calorie consumed by the user during the exercise by using the first feature information and the user's exercise duration to obtain a second estimation result;
outputting the second estimation result.

11. The method according to claim 10, further comprising:
generating and outputting encouragement information, if the evaluation score corresponding to the evaluation result is greater than a first preset threshold, and generating and outputting error warning information, if the evaluation score corresponding to the evaluation result is less than a second preset threshold.

12. The method according to claim 11, further comprising:
acquiring a user's playing instruction;
playing a media file within a preset historical time period comprising at least one of the following content according to the playing instruction: the image information, the evaluation result, the error warning information, and the encouragement information.

13. The method according to claim 11, further comprising:
generating an exercise report, comprising: the user's exercise duration, the first estimation result, the second estimation result, the evaluation result, the error warning information, and the encouragement information.

14. The method according to claim 13, further comprising:
acquiring a user's sharing instruction;
sending the exercise report to a preset terminal according to the sharing instruction.

15. The method according to claim 1, further comprising:
acquiring gesture information of a user;
analyzing the gesture information to obtain a control instruction corresponding to the gesture information;
executing the control instruction.

16. The method according to claim 1, further comprising:
acquiring second characteristic information of a user, comprising at least one of the following: heart rate information of the user, and breathing frequency of the user;
generating alarm information when a value corresponding to the second characteristic information exceeds a corresponding preset range of value;
outputting the alarm information.

17. A model establishing method, comprising:
obtaining a video of a training project;
processing the video by performing decomposition on the training actions to obtain frames corresponding to decomposed actions;
establishing an action model based on the frames corresponding to the decomposed actions, wherein the action model is used to perform evaluation on action information of at least one part of a user's body in collected image information containing a user's image.

18. The method according to claim 17, further comprising:
obtaining a set of samples;
using the set of samples to train the action model to optimize parameters in the action model.

19. The method of claim 17, wherein the establishing an action model based on the frames corresponding to the decomposed actions comprises:
performing identifying of a part of body on a frame to obtain an identifying result;
establishing sub-models corresponding to a plurality of parts of the body based on the identifying result;
obtaining the action model based on the sub-models corresponding to the plurality of parts of the body.

20. The method according to claim 17, further comprising:
storing a video of the training project in association with the action model.

21. A teaching device, comprising:
a collecting means configured to collect image information containing a user's image;
a processor configured to acquire an action model corresponding to training actions that the user refers to; perform evaluation on action information of at least one part of the user's body in the image information by using the action model to obtain an evaluation result; outputting the evaluation result to an outputting means.

22. A teaching system, comprising:
a teaching device configured to collect image information containing a user's images; and send the image information to a server;
a server configured to acquire an action model corresponding to training actions that the user refers to; perform evaluation on action information of at least one part of the user's body in the image information by using the action model to obtain an evaluation result; send the evaluation result to the teaching device,
wherein the teaching device is further configured to output the evaluation result on the action information of at least one part of the user's body.

23. An electrical apparatus, comprising: a memory and a processor, wherein
the memory is configured to store a program;
the processor is coupled to the memory, and is configured to execute the program stored in the memory, to: collect image information containing a user's image; acquire an action model corresponding to training actions that the user refers to; perform evaluation on action information of at least one part of the user's body in the image information by using the action model to obtain an evaluation result; output the evaluation result on the action information of at least one part of the user's body.

24. An electrical apparatus, comprising: a memory and a processor, wherein
the memory is configured to store a program;
the processor is coupled to the memory, and is configured to execute the program stored in the memory, to: obtain a video of a training project; process the video by performing decomposition on the training actions to obtain frames corresponding to decomposed actions; establish an action model based on the frames corresponding to the decomposed actions, wherein the action model is used to perform evaluation on action information of at least one part of a user's body in collected image information containing a user's image.
